(19) United States
(12) Plant Patent Application Publication
(10) Pub. No.: US 2017/0311504 P1
(43) Pub. Date: Oct. 26, 2017

(54) Broussonetia plant named 'Jinhudie'

(50) ;

(71) Applicant: HENAN MINGPIN COLORIZED SEEDLING CO., LTD, Suiping (CN)

(72) Inventor: Huaming Wang, Suiping (CN)

(21) Appl. No.: 15/731,043

(22) Filed: Apr. 11, 2017

(51) Int. Cl. A01H 5/12

(52) U.S. Cl. CPC A01H 5/12
USPC PLT/216

(57) ABSTRACT

‘Jinhudie’ is a distinctive variety of Broussonetia Plant, which is characterized by its color of leaves.

Genus and species: Broussonetia L'Hér. ex Vent.

Variety denomination: Jinhudie.

### BACKGROUND OF THE INVENTION

The present invention relates to a new and distinct variety of Broussonetia plant, which is given the varietal denomination of ‘Jinhudie’.

In April 2006, a mutated Broussonetia plant was found among Broussonetia seedlings in Chuxu Village, Suiping County, Henan Province, China. Leaf margins of the mutated Broussonetia plant were golden while leaf centers were green. The mutated plant was reproduced by budding with xylem.

In April 2007, the mutated Broussonetia plant was grafted with a rootstock of a wild Broussonetia plant and branches of the mutated plant. According to observation, in spring, leaf margins of new-born plant were golden while leaf centers were green. In summer, leaves turned to slight yellow-green with blackish green spots. In autumn, the leaf margins were orange-yellow while leaf centers were yellow-green.

In spring 2008, the grafted plant was asexually reproduced by grafting and cottage. Characteristics thereof were all kept.

From 2009-2013, after generations of reproducing, no atavism or further mutation was observed. Characteristics of the distinct variety were kept stable.

The new and distinct variety of Broussonetia plant is not commercially available.

### SUMMARY OF THE NEW VARIETY

‘Jinhudie’ is a bud mutation of Broussonetia plant with wide deciduous leaves, whose crown is extended and is oval-shaped or wide oval-shaped. Bark of ‘Jinhudie’ is smooth, light gray or grayish brown, and without cracking. The whole plant contains milk-like sap. Young branches are densely covered with fine hairs. Single-leaves, which grow alternately and sometimes almost oppositely, are thick papery. The leaves are oval-shaped or wide oval-shaped with acute apexes. Bases of the leaves are rounded, subcordate or skewed. The leaves have serrated margins, and have no crack or 3-5 cracks (more obvious on young branches). Upper surfaces of the leaves are rough and covered with hard hairs. Lower surfaces of the leaves are covered with soft hairs. There are 7-8 pairs of lateral veins. In spring, leaf margins are golden while leaf centers are green. In summer, leaves turned to slight yellow-green with blackish green spots. In autumn, the leaf margins were orange-yellow while leaf centers were yellow-green. Petioles long 3-5 cm, and are densely covered with thick hairs. Stipules are ovate or oblong, and caduceus. Flowers are female. Polythalamic fruits are spherical. Mature fruits are orange-red with nodules thereon. Florescence is from April to May while fruiting from August to September.

### BRIEF DESCRIPTION OF THE DRAWINGS

FIG. 1 is a color photograph of the new invention, ‘Jinhudie’, showing the plant in the field.

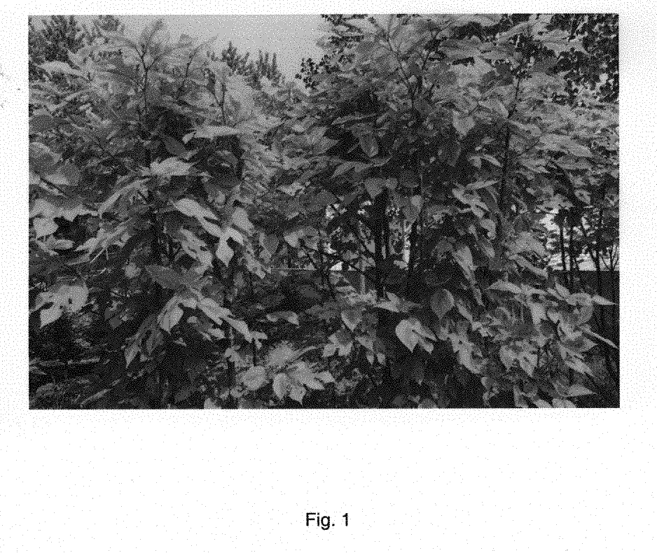

FIG. 2 is a color photograph of the new invention, ‘Jinhudie’, showing its stem.

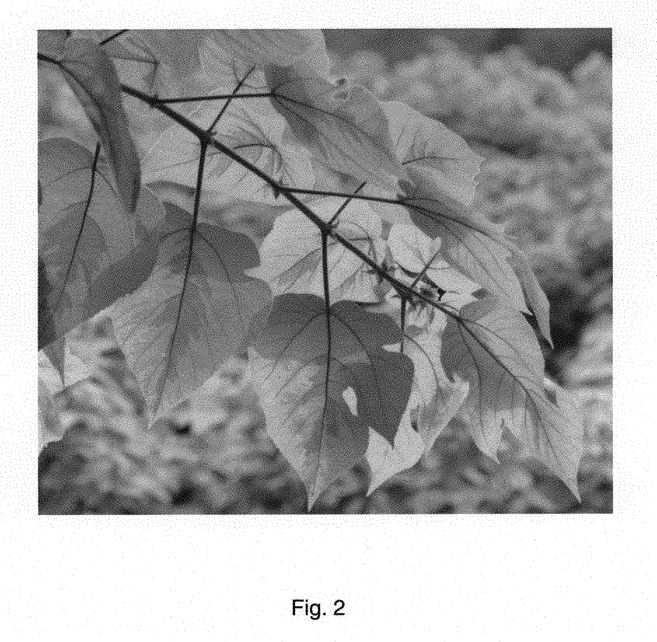

FIG. 3 is a color photograph of the new invention, ‘Jinhudie’, showing leaves in summer.

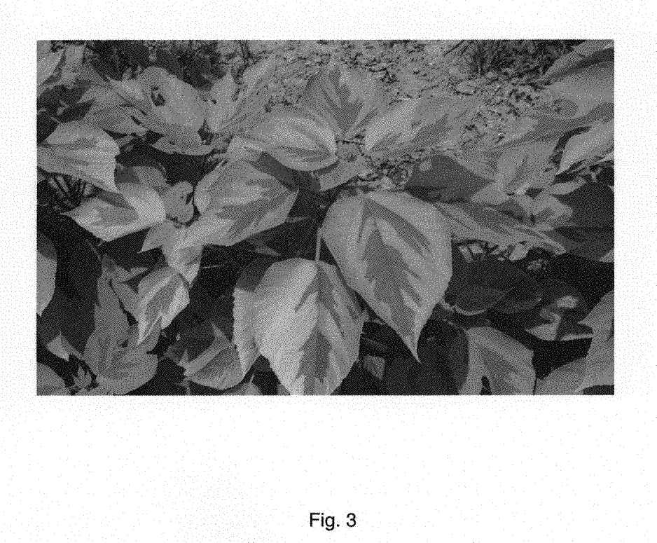

FIG. 4 is a color photograph of the new invention, ‘Jinhudie’, showing leaves in autumn.

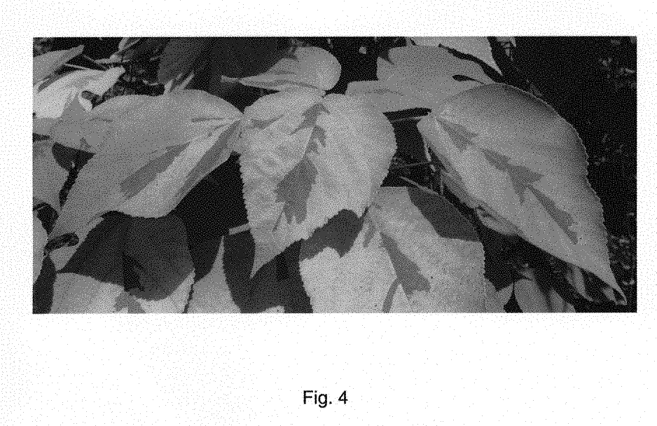

FIG. 5 is a color photograph of the invention, ‘Jinhudie’, showing the appearance of its flowers in spring.

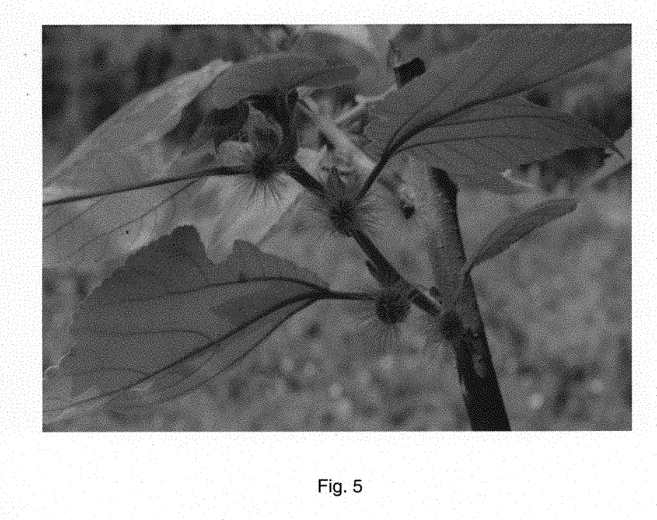

FIG. 6 is a color photograph of the invention, ‘Jinhudie’, showing the appearance of its fruits.

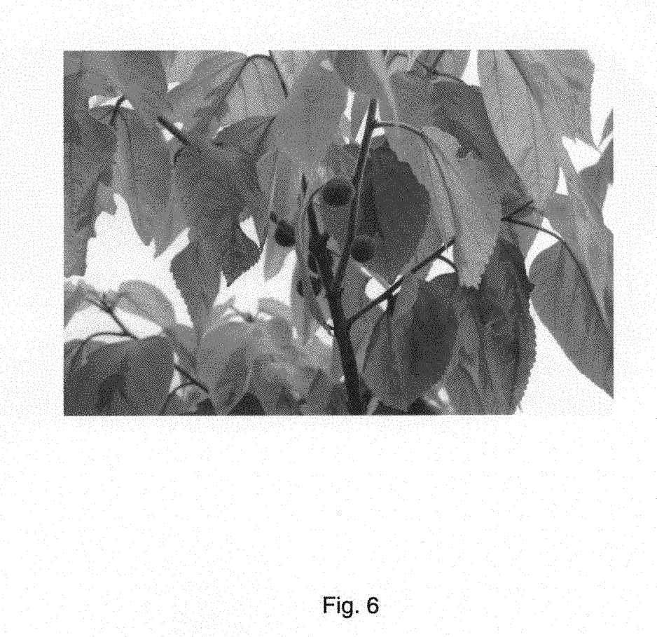

### DETAILED DESCRIPTION

The accompanying colored photographs illustrate the overall appearance of the new and distinct variety of ‘Jinhudie’. The descriptions disclosed herein are based upon observations of the plant growth.

‘Jinhudie’ is a bud mutation of Broussonetia plant with wide deciduous leaves, whose crown is extended and is oval-shaped or wide oval-shaped. Bark of ‘Jinhudie’ is smooth, light gray or grayish brown, and without cracking. The whole plant contains milk-like sap. Young branches are densely covered with fine hairs. Single-leaves, which grow alternately and sometimes almost oppositely, are thick papery. The leaves are oval-shaped or wide oval-shaped with acute apexes. Bases of the leaves are rounded, subcordate or skewed. The leaves have serrated margins, and have no crack or 3-5 cracks (more obvious on young branches). Upper surfaces of the leaves are rough and covered with hard hairs. Lower surfaces of the leaves are covered with soft hairs. There are 7-8 pairs of lateral veins. In spring, leaf margins are golden while leaf centers are green. In summer, leaves turned to slight yellow-green with blackish green spots. In autumn, the leaf margins were orange-yellow while leaf centers were yellow-green. Petioles long 3-5 cm, and are densely covered with thick hairs. Stipules are ovate or oblong, and caduceus. Flowers are female. Polythalamic fruits are spherical. Mature fruits are orange-red with nodules thereon. Florescence is from April to May while fruiting from August to September.

‘Jinhudie’ is a distinctive variety of Broussonetia plant, which is able to grow in north, center, south, southwest, and northwest of China, mainly in the Yellow River area, the Yangtze River area and the Pearl River Basin area. The plant grows better in the south of China.

‘Jinhudie’ is a strong phototropism tree species, whose adaptability is particularly strong. The plant has strong resistance to dry-cold and dry and damp-hot climates, and is able to grow in acidic soil, neutral soil and limestone mountain area due to resistance to drought and barren. The plant grows well at both waterside and wetlands. Roots of the plant are shallow and lateral, which are widely distributed and rapidly grow. ‘Jinhudie’ has strong abilities in budding and tillering, with strong resistance to pruning and pollution.

Reproducing methods of ‘Jinhudie’ are mainly graft and cottage. For grafting, whip, cleft and budding (with xylem) are mainly used, while tillering and laying reproducing are also acceptable. For grafting, new-born Broussonetia plant is used as a rootstock. In spring, cleft or budding with xylem should be adapted for reproducing. In summer, large-square budding is used for reproducing. For cottage, young summer branches are mainly used, wherein half-xylem young branch with a length of 10-15 cm is cut and inserted into seedling device. With a full-light cottage technology, rooting is extremely fast

‘Jinhudie’ can be cultivated and rapidly grow in both spring and autumn. During growth, trimming is needed. The plant is not suitable for growing at low areas which are like to suffer water logging.

‘Jinhudie’ rarely suffers from pests and diseases. However, in some particular regions having trunk-attacking pests such as longhorned beetles and jewel beetles, attention should be paid for preventing.

TABLE
comparison between ‘Jinhudie’ and similar species.

| similar | features of similar | |
|---|---|---|
| species | species | features of ‘Jinhudie’ |
| paper | dioecism | female |
| mulberry | single color | complex color |
| | young leaf is mainly | young leaf is mainly |
| | green | yellow |
| | no secondary color | secondary color is |
| | | green |
| | no distribution of | secondary color is |
| | secondary color | distributed at center |

These features and other characteristics of the plant are apparent from the figures.

What is claimed is:

1. A new and distinct variety of Broussonetia Plant named ‘Jinhudie’, substantially as described and illustrated.
